(19) United States
(12) Patent Application Publication
(10) Pub. No.: US 2026/0049357 A1
(43) Pub. Date: Feb. 19, 2026

(54) DNA BIOMARKER PANEL FOR THE DETECTION OF AUTISM SPECTRUM DISORDER

(71) Applicant: Clemson University Research Foundation, Clemson, SC (US)

(72) Inventors: Frank A. Feltus, Clemson, SC (US); Rini Pauly, Frederick, MD (US); Emily Casanova, New Orleans, LA (US)

(21) Appl. No.: 19/294,554

(22) Filed: Aug. 8, 2025

Related U.S. Application Data
(60) Provisional application No. 63/681,211, filed on Aug. 9, 2024.

(51) Int. Cl. C12Q 1/6883

(52) U.S. Cl. CPC C12Q 1/6883; C12Q 2600/156

(57) ABSTRACT

Methods of identifying elevated levels of one or more biomarkers in a subject for detecting or diagnosing autism in subject is provided. The one or more biomarkers have been identified as biomarkers being elevated in autism. The methods include determining the levels of one or more biomarkers in a biological sample from a subject, wherein the one or more biomarkers comprise one or more single nucleotide polymorphisms (SNPs) found Cox10, RAD1, DKK3, STK32C, AARS2, POLR1C, or a combination thereof. The elevated levels of one or more of these biomarkers may be an indicator of autism in the subject.

### RELATED APPLICATIONS

This application claims priority to U.S. Provisional Patent Application No. 63/681,211, filed Aug. 9, 2024, the entire contents of which are incorporated herein by reference.

### TECHNICAL FIELD

This disclosure relates to biomarkers for use in the detection of autism spectrum disorder, including the use of Neanderthal single nucleotide polymorphisms (SNPs) that are enriched in autism.

### BACKGROUND

Approximately forty-seven to sixty-five thousand years ago (KYA), ancient Homo sapiens interbred with Neanderthals as the former group migrated out of Africa into the Levant region and into the farther reaches of Asia and Europe. Due in part to population bottlenecks in both of these early groups of closely related humans, Neanderthal-derived DNA successfully spread through the early Eurasian population, such that all modern Eurasian people maintained a small but significant proportion of Neanderthal DNA. About 20 KYA, a subset of Europeans migrated back into Africa, leading to indirect introgression of Neanderthal DNA into the African populations. Thus, all modern people carry the genetic signature of these ancient hybridization events to varying degrees.

DNA evidence taken from human remains from the Middle Pleistocene indicate that anatomically modern humans (AMH) and other archaic humans underwent multiple introgression events. Of those archaic humans, Homo neanderthalensis (Neanderthals) has received the most attention, providing the most fossil material and occupying the position as our closest known cousin on the hominin tree of life. It has been estimated that Eurasian-derived populations have approximately 2% Neanderthal DNA, which was acquired during introgression events occurring shortly after AMH migrated out of Africa. These hybridization events occurred somewhere between 47-65 thousand years ago (kya). A subset of Europeans later immigrated back into Africa approximately 20 kya, bringing some of this Neanderthal ancestry with them, such that all modern Africans have a small but measurable amount of Neanderthal DNA from the event.

With the recent sequencing of multiple archaic human genomes, there has been growing interest concerning the influence of archaic human-derived alleles on modern health. With regards to Neanderthal-derived variants, previous groups have identified positive selection on genes relating to immune function, skin and hair pigmentation, physiological responses to high altitude conditions, aspects of metabolism, hypercoagulation, and propensity for depression.

In general, dosage-sensitive genes are tightly conserved and resistant to such introgression events. Most genes involved in brain development follow this dosage-sensitive pattern and have tended to be resistant to introgression. In support of this, Srinivasan et al. found a depletion of Neanderthal-derived variants within autism-and other brain-related genes in the general population. On the other hand, other studies have reported a number of non-synonymous and other single nucleotide variants within neural genes that have been implicated in the condition.

Additional research involving non-clinical populations has identified strong links between certain brain and skull morphologies and enrichment of Neanderthal DNA. Specifically, enrichment is associated with reduced globularity in the skull shape of modern populations, a finding mildly reminiscent of the elongated skull morphology characteristic of Neanderthal and other archaic crania. Enrichment of Neanderthal DNA is also associated with enhanced neural connectivity within visual processing systems, particularly between the intraparietal sulcus (IPS) and the occipital cortex and fusiform gyrus, and decreased connectivity within the default mode (social) network. There remains a need for identifying genes associated with the development of autism, as well as diagnostics for early identification of autism in a subject.

### BRIEF DESCRIPTION OF DRAWINGS

FIG. 1 is a diagram of group comparison of SNP Frequencies and Quantitative Trait Loci (QTL) Network. FIG. 1A shows the rare and common SNP frequencies by ethnic control group. FIG. 1B is a bar plot with comparative Total NeanderScores between controls and autism broken down by ethnicity. No significant differences between autism and controls except in the white Hispanic (WHS) group. FIG. 1C is a diagram of common NeanderScores across experimental and control groups by ethnicity. In general, autism groups—with the exception of white, non-Hispanics (WNH)—have lower common NeanderScores. FIG. 1D is a diagram of NeanderScores of autism and controls by ethnicity. Note that all autism groups have significantly higher Rare NeanderScores compared to controls. FIG. 1E is a cytoscape network illustrating clinical associations in autism (white nodes) with various brain-associated QTLs (shaded nodes) that were either rare in autism or significantly enriched. SNPs are color-coded according to ethnic group. (Salmon=BNH, green=WHS, blue=WNH).

FIG. 2 is a bar graph showing differences in frequencies of target SNPs in Black American autistic and non-clinical control samples.

FIG. 3 is a Forest plot illustrating the odds ratios of SNPs of interest between autistic and control groups.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular methods which can vary and are understood by skilled artisans. It is further to be understood that all terminology used herein is for the purpose of describing particular embodiments only and is not intended to be limiting in any manner or scope. So that the present disclosure may be more readily understood, certain terms are first defined.

Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The term “about,” as used herein, refers to variation in the numerical quantity that can occur, for example, through typical measuring techniques and equipment, with respect to any quantifiable variable, including, but not limited to, mass, volume, temperature, and time. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

The methods and compositions of the invention relate to the roles of genic Neanderthal-derived single nucleotide polymorphisms (SNP) in autism susceptibility. Although long-conserved sequences are presumed essential or advantageous, in some aspects, a subset of rare and uncommon SNPs that influence risk across a substantial minority of the autistic population have been identified and developed as genomic biomarkers for diagnostics. while common SNPs do not appear to play a measurable role. Therefore, in some aspects, although individuals with autism do not have more Neanderthal DNA compared to the general population, they do, on average, have more rare and uncommon Neanderthal DNA content, as do their unaffected siblings.

Provided within the disclosure are methods of identifying subsets of rare and uncommon Neanderthal-derived genic variants enriched in subjects with autism. In some aspects, 52 specific genic Neanderthal-derived single nucleotide polymorphisms (SNPs) are implicated in autism. Within the disclosure, further SNP patterns have been identified to be implicated in autism. For example, in some instances, SNP patterns have been identified as being present in over 7% of European American autistic cases that are rare in race-matched controls, a figure comparable to the rates of fragile X syndrome in autism.

In some aspects, the disclosure provides for methods of diagnosing autism in a subject. In further aspects, the disclosure provides for methods of identifying elevated levels of a biomarker. In some implementations, the methods may comprise determining the levels of one or more biomarkers in a biological sample from a subject. In some aspects, the elevated levels of one or more biomarkers may be an indicator of autism in the subject.

In embodiments, the biomarker for use in the disclosed methods comprise SNPs enriched in subjects with autism and spread across multiple genes. Not to be bound by any theory or single function of any specific gene, SNPs included in some of the embodiments are found in genes that serve diverse functions in the body. Mutations in genes such as AARS2 and POLRIC are associated with leukodystrophies, which are a group of genetic disorders affecting the white matter of the brain. The STK32C gene is a member of the serine/threonine protein kinase family and is thought that it is functional in the brain due to its high expression levels therein. The COX10 encodes a protein that is essential for the function of cytochrome c oxidase, a key enzyme in the mitochondrial respiratory chain. The RAD1 gene encodes a protein that is a component of the 9-1-1 complex, a crucial player in DNA damage response and repair. The DKK3 gene, also known as Dickkopf WNT signaling pathway inhibitor 3, encodes a secreted protein that plays a role in various biological processes, including development, tissue homeostasis, and disease pathogenesis.

In further embodiments, additional associations with sex and language delay have been identified in African American individuals with autism. In some cases, the biomarkers are found in the RAD1 variant, such as, for example, rs1805327 SNP. In some aspects, although the rs1805327 SNP is a common minor allele in Eurasians, it is generally rare in African populations according to the 1000 Genomes Project (minor allele frequency: 0.002), suggesting it may be a significant autism susceptibility factor when present upon these genetic backgrounds. Since the RAD1 protein is part of the 9-1-1 complex, its cognate partners, RAD9 and HUS1, may be additional targets across these ethnic backgrounds. Therefore, in some aspects, the biomarker may comprise an SNP found in RAD1, RAD9, HUS1, or a combination thereof.

In embodiments, the methods of the disclosure may be used to detect biomarkers for the diagnosis of autism in a subject. In some aspects, the subject is suffering from symptoms of autism. In other aspects, the subject may not show any symptoms of autism. In embodiments, the methods of the disclosure may be administered to subjects who are European American or African American.

In some aspects, the methods of the disclosure may be administered in addition to another diagnostic test or therapeutic treatment. The methods of the disclosure may be administered at any time to the subject. In some aspects, the methods may be administered in utero or ex utero. In other aspects, the methods may be administered to a subject less than one year of age, one year or age or older, 5 years of age or older, 10 years of age or older, 15 years of age or older, 20 years of age or older, 25 years of age or older, 30 years of age or older, 35 years of age or older, 40 years of age or older, 45 years of age or older, or 50 years of age or older.

In some aspects, the methods of the disclosure may be administered at least one time per day, at least one time per week, at least one time per month, at least one time per year, or at least time to a subject in need thereof. More frequent testing may be completed as needed depending on the needs of the subject.

In some aspects, the SNPs identify locations for potential gene therapy or gene editing. For example, functional genomics tools such as CRISPR, base editing, siRNA or antisense could be utilized to alter the base at the SNP location to alter the phenotype of the gene.

In another aspect, a gene panel can be designed to comprehensively diagnose a subject. The panel can be comprised of 2, 3, 4, 5 or more markers. In another aspect, the SNPs disclosed herein are combined with other SNPs identified to be associated with Autism. In another aspect, the panel is a subpanel of a larger panel identifying a number of conditions. In another aspect, the SNPs can be identified through partial, exome or whole genome DNA sequencing of a subject.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Enrichment of Rare and Uncommon Genic Neanderthal-Derived SNPs in Autism and Unaffected Siblings

An assemblage of European lineage-specific Neanderthal-derived single nucleotide variants (GRCh37) was provided by the Sankararaman laboratory for analyses on European American and African American participants. Whole exome sequencing (WES) and other demographic and clinical data were accessed from the Simons Foundation Powering Autism Research (SPARK) database where DNA polymorphisms were mapped to the GRCh38 human genome assembly. Race-matched controls were derived from the GTEx Project (GRCh38). LiftOver was used to convert Neanderthal SNP GRCh37 positions to GRCh38. The basic demographics of the participants in the Example can be found below in Table 1.

TABLE 1
Basic demographics of participants

| Ethnic group | Group | Total number (N) | Males | Females |
|---|---|---|---|---|
| Black, Non- | Control | 102 | 70 | 32 |
| Hispanic (BNH) | Autism | 72 | 61 | 11 |
| White Hispanic | Control | 74 | 40 | 34 |
| (WHS) | Autism | 312 | 255 | 57 |
| White, Non- | Control | 706 | 465 | 241 |
| Hispanic (WNH) | Autism | 2004 | 1567 | 437 |
| | Siblings | 172 | 142 | 30 |

Genotype calls were processed from SPARK and GTEx VCFs by converting genotypes into a binary call format and then transformed into genotype matrices using JVARKIT. Subsequently, all SNPs with missing data points were removed from the analysis, resulting in a total of 1,288 SNPs for comparison. These SNPs were then categorized as “rare” (<1%), “uncommon” (1%≤×>5%), and “common” (≥5%) in the European American groups and “rare” (<1%) and “not rare” (>1%) in the African American groups according to their frequencies within their respective control groups. A binary approach was used for ranking within the African American groups due to overall low frequency of Neanderthal-derived SNPs compared to European Americans.

The embodiments of the invention demonstrate the use of the detection of Neanderthal DNA that is enriched in autistic people and their siblings compared to ethnically-matched controls. Whole exome sequencing (WES) for autistic probands and unaffected siblings was accessed from the Simons Foundation Powering Autism Research (SPARK) Database for comparison against individuals in the Genotype-Tissue Expression (GTEx) and 1000 Genomes (1000G) databases. Significant enrichment in the autism group was especially driven by rare Neanderthal-derived variants, but also some common variants, which suggests weak but ongoing purifying selection towards removal of some of these single nucleotide polymorphisms (SNP) from the human genome.

### Example 2

### Selection of Genetic Markers

### Analysis of African American Groups

Participant numbers in the African American samples were small compared to the European American groups. However, one SNP in particular, at locus Chr5: 34908772:T>C (rs1805327) and housed within the RAD1 gene, remained significant following multitest correction. This SNP produced a missense variant, changing a glutamic acid to a glycine, a substitution which is generally disfavored amongst intracellular proteins such as RAD1. This SNP occurred in 9.3% of the autism sample compared to no instances within the controls or unaffected sibs. It was discovered that individuals positive for this variant did not differ in overall NeanderScores (total Neanderthal content), suggesting that this enrichment was a result of clinical phenotype and not due to higher European admixture (W=260.500, p=0.6884). Further, this SNP is not rare in European Americans, suggesting it may be an African American-specific susceptibility factor.

In further analyzing this polymorphism, this variant shares an interesting association with sex: females are equally as likely to carry the variant as males, which is different from the typical extreme male sex-skewing present in autism, including within this sample (X²=4.144, p=0.0418). Twenty-five percent of females within the Example (3 out of 12) carried the variant, and 1 girl was even homozygous. This was compared to only 6% of males (4 out of 63). All 4 males with this RAD1 variant were reported to have had language delay; meanwhile, none of the females had this clinical feature, including the homozygous female. While due to small numbers in this initial example, this result did not reach significance compared to the non-RAD1 subset, it was trending in that direction (H=7.612, df=3, p=0.0548).

### Example 3

### Enrichment of a Subset of Neanderthal Polymorphisms in Autistic Probands and Siblings

Homo sapiens and Neanderthals underwent hybridization during the Middle/Upper Paleolithic age, culminating in retention of small amounts of Neanderthal-derived DNA in the modern human genome. The current example addresses the potential roles Neanderthal SNPs may be playing in autism susceptibility in samples of black non-Hispanic, white Hispanic, and white non-Hispanic people using data from the Simons Foundation Powering Autism Research (SPARK), Genotype-Tissue Expression (GTEx), and 1000 Genomes (1000G) databases. Based on the results of this example, it has been discovered that rare variants are significantly enriched in autistic probands compared to race matched controls. In addition, 25 rare and common SNPs have been identified herein that are significantly enriched in autism on different ethnic backgrounds, some of which show significant clinical associations. Other SNPs have also been identified herein that share more specific genotype-phenotype correlations but which are not necessarily enriched in autism and yet may nevertheless play roles in comorbid phenotype expression (e.g., intellectual disability, epilepsy, and language regression). The results in this example strongly suggest Neanderthal-derived DNA is playing a significant role in autism susceptibility across major populations in the United States.

An assemblage of Neanderthal-derived SNPs was provided by the Sankararaman laboratory and has been previously published. Only European-specific SNPs were used in the current study due to European admixture across these various ethnic groups. Access was granted to WES from the SPARK dataset (Clemson University IRB2018-235), which is a collection of extensive genotype and clinical data on autistic individuals and their unaffected siblings. Samples have been collected from a wide range of collection sites within the US and can be found on the SFARI website (see: https://www.sfari.org/resource/spark/). Only individuals of self-reported “white” (white Hispanic, white non-Hispanic) and “black” (black non-Hispanic) backgrounds were used in these analyses; any participant listed as “admixed” was removed. Autistic individuals with potential confounding genetic and environmental factors were also excluded from the study. These conditions include: congenital anomalies, birth or pregnancy complications, premature birth, fetal alcohol syndrome, and cognitive delays due to exposure or medical condition. Ethnically matched controls were accessed from the GTEx Project (Clemson University IRB2022-0589) for comparison with the black non-Hispanic and white non-Hispanic SPARK groups respectively. GTEx was determined to include too small of a sample (<2%) of Hispanic individuals for adequate comparison, so these were removed from the GTEx sets and a different control group was used for white Hispanics in the SPARK group. For white Hispanics, the Mexican (MXL) and Puerto Rican (PUR) ancestry subgroups from 1000G were used as controls—data that are freely available for download. According to the Pew Research Center, people of Mexican ancestry currently compose about 60% of all Hispanics within the mainland US, while Puerto Ricans compose another 9%. Together, these two groups of Hispanics compose the largest subset of Hispanic people in the mainland US and are available in 1000G, and for this reason these subgroups have been selected to approximate a control group, utilizing a 1:6 (PUR:MXL) ratio in order to best mimic the Pew data.

Allelic frequencies were determined using ethnically matched control groups. While numbers of white non-Hispanic exomes were sufficient (GTEx N=706, SPARK affected=2004, SPARK unaffected siblings=172), the number of black non-Hispanic SPARK affected and GTEx individuals were comparatively small (SPARK N=72, GTEx N=102), which limited the analysis of this latter subset (see Table 1 for basic demographics). In addition, the white Hispanic control group was similarly limited (N=74), which has likely affected the Rare NeanderScore results, creating the appearance of an even greater divergence than may exist. All SNPs with missing data were removed from the analyses due to significant fluctuations in results when even small amounts of missing data were retained (e.g., even a 5% threshold for missing data dramatically altered results with the current study design). After removal of 546 SNPs with missing data, this left a total of 1288 SNPs for comparison across all groups.

To process the genotype calls from SPARK, GTEx, and 1000G VCFs, the individual genotypes were converted into a binary call format (bcf) using bcftools (see: https://github.com/samtools/bcftools). Subsequently, the bcf files were transformed into genotype matrices using the JVARKIT tools developed by Pierre Lindenbaum. The JVARKIT tools can be found at the following GitHub repository (see: https://github.com/lindenb/jvarkit/).

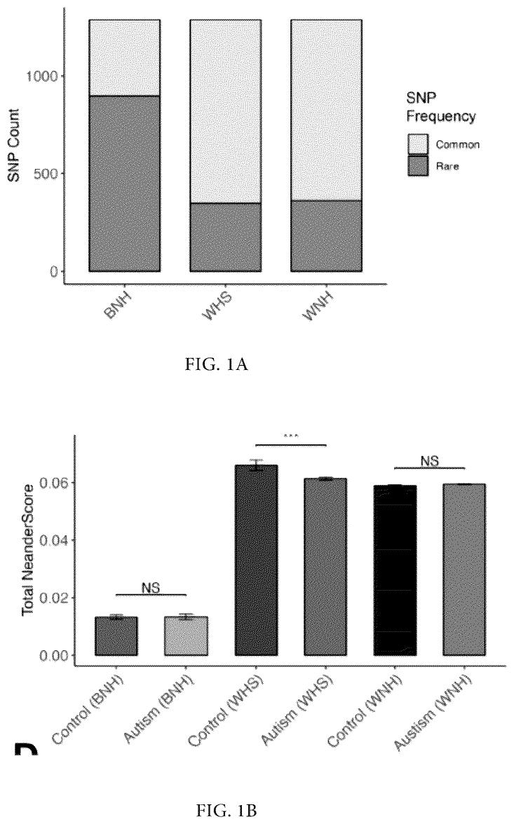

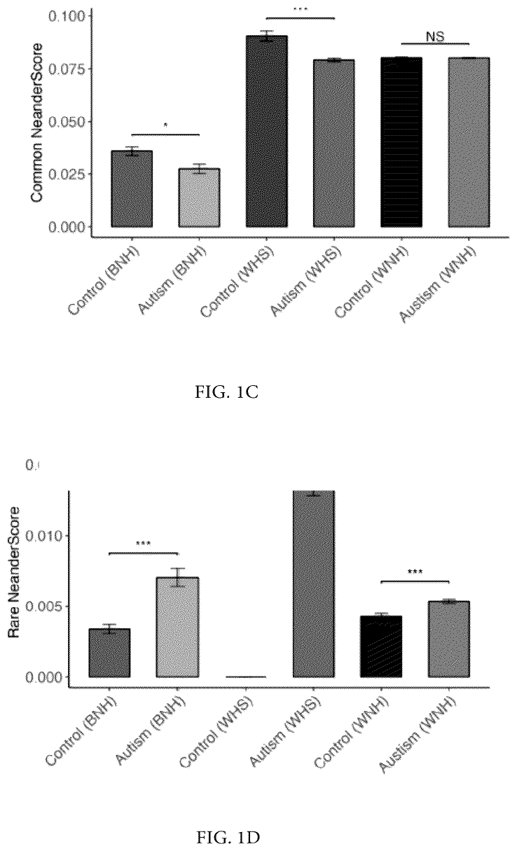

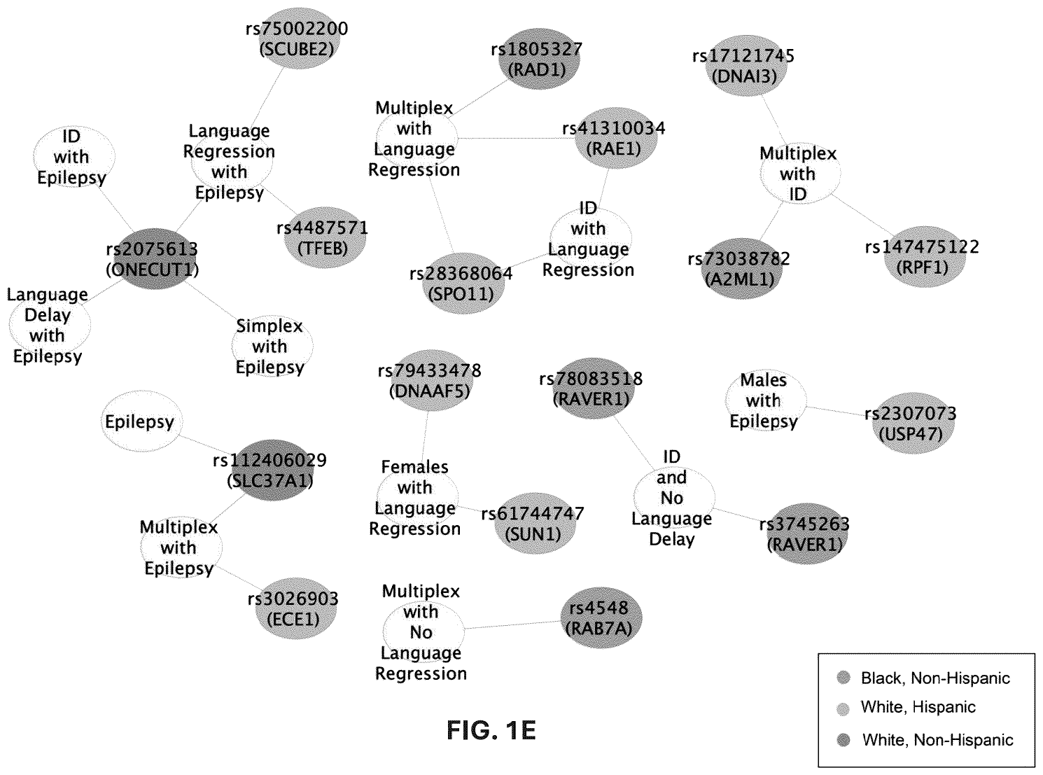

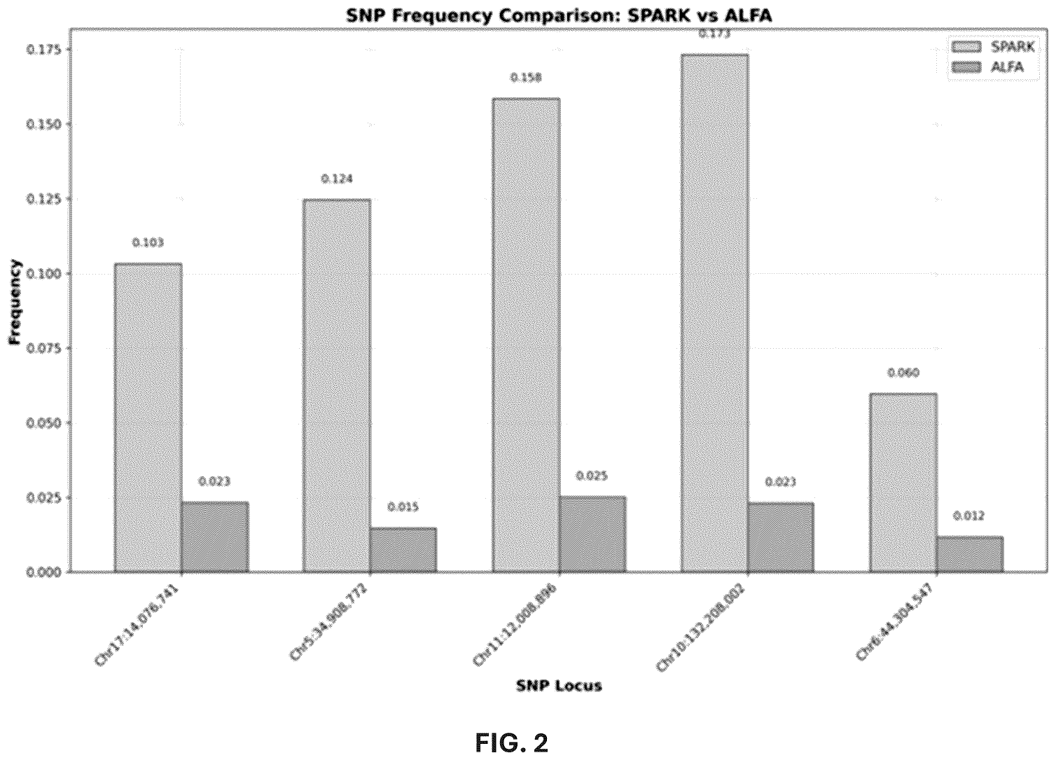

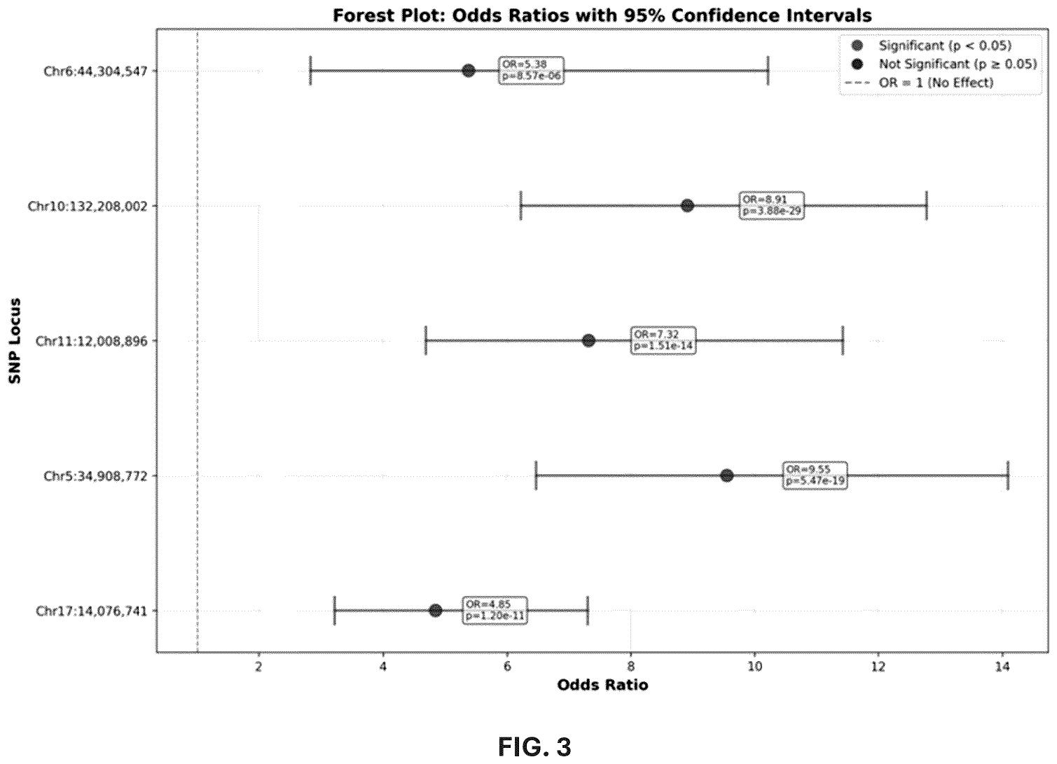

Autism SNPs were categorized as “rare” (<1%) and “common” (≥1%), according to their frequency within the respective GTEx/1000G control datasets [27] (see FIG. 1A for rare-to-common SNP ratios by ethnic control group). Across all groups, homozygous ancestral (non-Neanderthal) SNPs were assigned a value of “0,” heterozygous Neanderthal SNPs were given a value of “1,” and homozygous Neanderthal alleles assigned the value of “2.” Raw NeanderScores were then calculated according to the average of these values per individual (divided by total possible SNPs, e.g., 1288 for Total NeanderScores) and larger group averages were subsequently broken down into the rare and common categories for additional analysis. The term, “NeanderScore,” is used here to refer to the average SNP enrichment in any given individual, with “Rare, “Common,” and “Total” NeanderScores referring to averages across groups of SNPs with varying frequencies. For instance, a given individual's “Rare NeanderScore” within the white non-Hispanic group is calculated by tallying the total number of hits per person (0=homozygous ancestral, 1=heterozygous Neanderthal, 2=homozygous Neanderthal) across the 348 rare SNPs and then averaged by dividing with the total number of possible hits (i.e., 348). This provides a percentage of enrichment, which, along with other individuals' Rare NeanderScores, can then be used to determine group means and variance for comparison.

For enrichment analyses, all SNPs enriched by ≥5% in either the black non-Hispanic or white non-Hispanic autism groups relative to controls, or enriched by ≥10% in the white Hispanic autism group, were selected for additional investigation. The white Hispanic cutoff was set higher relative to the other two ethnic groups due to greater deviation between white Hispanic autism and control data, which is likely due to poorer match of the control group such that a more conservative cutoff was deemed warranted (see FIG. 1B). SNPs that achieved these cutoffs were statistically analyzed for enrichment relative to controls using two-proportion Z tests with Benjamini-Hochberg (BH) multitest correction. Finally, all SNPs that retained significance after correction but were not expressed in brain according to GTEx's Locus Browser (Variant-centric), which allows visualization of QTLs by tissue type, were also removed. This resulted in a final list of 6 brain-related QTLs in 5 host genes that were significantly enriched in the black non-Hispanic autism group, 18 SNPs in 15 host genes in the white Hispanic group, and 1 SNP in the white non-Hispanic group (25 in total). Gene consequences were derived from dbSNP. Using NIH's LDexpress tool, a list of host genes with SNPs in significant linkage disequilibrium in brain with the 25 SNPs of interest was also produced. The standard settings were used, which included a window of 500 k bp and R2>0.1, and all brain-related tissues were selected prior to search, such as “Brain-Cortex,” “Brain-Cerebellum,” etc. For each of the 6 SNPs enriched in black non-Hispanics, African Ancestry in Southwest USA (ASW) was used as a population background, as not all SNP pairings share LD on all genetic backgrounds. Meanwhile, both the MXL and PUR populations were used as a combined background for the 18 white Hispanic SNPs. And, finally, Northern Europeans from Utah (CEU) were used as background for white non-Hispanics. This resulted in a list of 43 host genes with additional brain-related QTLs of potential interest.

The original 25 SNPs were then analyzed for clinical associations available via the SPARK dataset. All other rare SNPs not significantly enriched in the autism groups were also assessed for clinical associations using t-tests/Mann-Whitney U tests and ANCOVAs, as they may still play important roles in comorbid clinical phenotypes despite a lack of relative enrichment compared to controls. Only SNPs that were brain-related QTLs were retained for the production of the genotype-phenotype Cytoscape network.

Finally, all rare SNPs enriched (not necessarily significantly) in the respective autism groups were analyzed for various functional enrichment patterns (e.g., Gene Ontology, KEGG Pathway, REAC, transcription factor binding sites) using the g: Profiler platform, which provides a list of significant enrichment terms as well as FDR-adjusted p-values.

For NeanderScores that followed a normal distribution, parametric statistics were used (i.e., t-test, ANOVA), with standard post hoc comparison when appropriate. Meanwhile, for datasets that were skewed (Shapiro-Wilk) or displayed unequal variances (Levene's), nonparametric analyses such as Mann-Whitney U, Welch t-test, and Kruskal-Wallis were used, with Games-Howell post hoc comparison when appropriate. For binomial data (e.g., presence/absence), Chi-square and odds ratios were used. For covariates such as “Language Regression×Epilepsy,” ANCOVAs were used. Means and standard deviations for each analysis are available in the Supplementary Materials (which are provided at https://www.nature.com/articles/s41380-024-02593-7 #Sec10, which is incorporated herein by reference in its entirety), as are all statistical analyses. Analyses were conducted and plots produced using R and Cytoscape, the former with the ggplot2 package.

Within the present work, “NeanderScore” refers to an average of the Neanderthal DNA content within a given person's genome, calculated based on the total number of Neanderthal-derived SNPs surveyed [7, 24]. NeanderScores were further divided into “Rare” and “Common” for additional analyses. All NeanderScores for the current study were calculated using only genic content and did not analyze intergenic regions.

Total NeanderScores differed significantly between ethnic groups as expected, with NeanderScores highest in white Hispanic people, followed by white non-Hispanics, and finally black non-Hispanics (H=525.172, p=2.939×10−111, η2 =0.532] (FIG. 1B). However, according to posthoc analysis, Total NeanderScores did not differ between black non-Hispanic and white non-Hispanic experimental and control groups, although white Hispanic controls had significantly higher Total NeanderScores than their SPARK counterparts, which may be a reflection of small control numbers (p=0.004).

Rare SNPs occur, by definition, in less than 1% of the general population, while common in ≥1% of the population. Within each ethnic grouping, SNPs were broken down into these two categories (rare and common) according to their frequency within the GTEx or 1000G control groups and analyzed accordingly. Utilizing this approach, potential enrichment of common Neanderthal-derived SNPs was investigated in the autism groups compared to ethnically matched controls. Both the black non-Hispanic and white Hispanic autism groups exhibited significantly lower Common NeanderScores (i.e., had fewer common Neanderthal variants) (black non-Hispanic: W=4617.500, p =0.004; white Hispanic: t(88.520)=−4.355, p =3.568×10-5). Meanwhile, autistic white non-Hispanics and their sibs did not significantly differ from the control group nor each other (F(2, 2879)=2.225, p=0.108) (FIG. 1C).

In contrast to Total and Common NeanderScores, it was apparent there was a dramatic enrichment of rare SNPs in all SPARK groups compared to ethnically matched controls (black non-Hispanics: H=1767.500, p=5.308×10−9; white Hispanic: X2=281.994, OR =7.624, 95% CI (4.809, 10.440), p=3.511 ×10−61; white non-Hispanic post hoc: t=−3.997, pTukey=1.940×10−4) See FIG. 1D.

According to Standard post hoc comparisons, white non-Hispanic autistic people did not significantly differ from sibs (ptukey=0.746) and sibs did not significantly differ from controls (pTukey=0.354) as their scores hovered midway between the other two groups. Unfortunately, sibling numbers were too low in the other two ethnic groups to enable similar comparisons. Lastly, sex was not a significant predictor of rare SNP enrichment within the various autism groups (p=0.271-0.830), although males in general tended to have higher scores.

In order to further test the validity of the rare SNP categorization, a randomization test was performed on white non-Hispanic rare SNPs as this autism group displayed the smallest divergence from controls. The difference of the means of Rare NeanderScores between autism and controls was 1.086×10−3. After 10,000 randomized repetitions, the average difference of the means was 4.191×10−6, which is significantly less than the observed difference (p=1.000×10−4), indicating that the original result was not obtained at random and lends further support to these results.

Following these analyses, functional enrichment studies were performed on rare SNPs enriched (though not necessarily significantly) in each autism group using the g: Profiler platform. Across all three ethnic groups, terms relating to “cytoskeleton” and/or “cell projection” were over-represented in the rare enriched genes (see Table 2). Within the white Hispanic rare genes, terms such as “nervous system development” and “neurogenesis” were also over-represented.

TABLE 2
Significant trends in functional enrichment of SNPs across
the three ethnic groups according to g:Profiler.

| Group | Category | Term name | Term ID | P(ad]) |
|---|---|---|---|---|
| BH (217 | GO: biological | Cytoskeleton organization | GO: 0007010 | 3.959 × |
| genes) | process | | | 10−2 |
| | GO: cellular | Plasma membrane bounded cell projection | GO: 0120025 | 4.830 × |
| | compartment | | | 10−3 |
| | | Cytoskeleton | GO: 0005856 | 1.794 × |
| | | | | 10−2 |
| | | Axoneme | GO: 0005930 | 1.794 × |
| | | | | 10−2 |
| | REAC | Signaling by cytosolic FGFR1 fusion | REACR-HSA- | 4.935 × |
| | | mutants | 1839117 | 10−2 |
| | GO: biological | Anatomical structure development | GO: 0048856 | 3.713 × |
| | process | | | 10−9 |
| WHS | | Cell-cell adhesion | GO: 0098609 | 7.508 × |
| (199 | | | | 10−5 |
| genes) | | Nervous system development | GO: 0007399 | 3.396 × |
| | | | | 10−4 |
| | | Plasma membrane bounded cell projection | GO: 0120036 | 1.984 × |
| | | organization | | 10~3 |
| | | Neurogenesis | GO: 0022008 | 1.809 × |
| | | | | 10−2 |
| | GO: cellular | Cell projection | GO: 0042995 | 3.126 × |
| | compartment | | | 10−3 |
| | | Cytoskeleton | GO0005856 | 1.542 × |
| | | | | 10−2 |
| WNH | GO: biological | Homophilic cell adhesion via plasma | GO: 0007156 | 9.939 × |
| (171 | process | membrane adhesion molecules | | 105 |
| genes) | | Anatomical structure development | GO: 0048856 | 1.106 × |
| | | | | 10−3 |
| | | Cell adhesion | GO: 0007155 | 1.298 × |
| | | | | 10−3 |
| | | Developmental process | GO: 0032502 | 9.000 × |
| | | | | 10−3 |
| | | Cell junction organization | GO: 0034330 | 2.743 × |
| | | | | 10−2 |
| | | Negative regulation of multicellular process | GO: 0051241 | 4.845 × |
| | | | | 10−2 |
| | GO: cellular | Cell projection | GO: 0042995 | 5.384 × |
| | compartment | | | 10−3 |
| | | Membrane | GO: 0016020 | 2.815 × |
| | | | | 10−2 |
| | | Plasma membrane bounded cell projection | GO: 0120025 | 2.817x × |
| | | | | 10−7 |
| | | Cell junction | GO: 0030054 | 4.430 × |
| | | | | 10−2 |

Next, significant enrichment of specific SNPs was investigated across the different ethnic groups. A total of 6 SNPs in the black non-Hispanic autism group, 18 SNPs in the white Hispanic group, and 1 SNP in the white non-Hispanic group were significantly enriched relative to controls and, according to the GTEx's Variantcentric Locus Browser, are brain-associated QTLs (see Table 3). Numbers of black non-Hispanic sibs were too small for analysis; however, white Hispanic sibs showed a similar enrichment as their affected siblings and did not significantly differ from each other with the exception of a single SNP (rs117034642), which was even higher in unaffected sibs (Z=−2.776, BH adj. p=0.049). There was no enrichment difference between autistics and sibs regarding the single enriched SNP in the white non-Hispanic group (Z=0.759, p=0.447). These 25 SNPs were then fed into NIH's LDexpress tool, which resulted in a further 43 host genes containing brain related QTLs in significant LD with the original SNPs of interest. Interestingly, within the white Hispanic autism group, there was a notable enrichment, not only of heterozygous variants in the 18 SNPs overrepresented in this group, but of homozygous variants as well (heterozygous: W=17,822.500, p=2.146×10−13; homozygous: W=13,761.000, p=1.878×10−4). Regarding the single SNP enriched in white non-Hispanics, this same trend was not seen (p=0.174). This analysis was not performed on the black non-Hispanic group as none of the 6 SNPs occurred in homozygous form in this group.

TABLE 3
List of Neanderthal-derived brain-related quantitative trait
loci (QTL) enriched in different ethnicities in autism.
Table 3. SNP Enrichment by Ethnic Group

| | | | | | |
|---|---|---|---|---|---|
| | | Frequency | | | Host genes |
| | | in | Host | Consequence | with SNPs |
| Locus | Rs ID | Controls | gene(s) | (dbSNP) | in LD |

Black, Non-Hispanic (BNH)

| | | | | | |
|---|---|---|---|---|---|
| 10: 132208002: G > A | rs79220014 | Rare | STK32C | STK32C: Non | DPYSL4 |
| | | | | Coding | JAKMIP3 |
| | | | | Transcript | |
| | | | | Variant | |
| 17: 14076741: A > T | rs2230351 | Rare | COX10 | COX10: | CDRT15 |
| | | | | Missense | |
| | | | | Variant (T > S) | |
| 5: 34908772: T > C | rs1805327 | Rare | RAD1 | RAD1: | RAI14 |
| | | | | Missense | |
| | | | | Variant (E > G) | |
| | | | | TTC23L: | |
| | | | | Intron Variant | |
| 11: 12008896: C > G | rs28411401 | Rare | DKK3 | DKK3: Intron | MICAL2 |
| | | | | Variant | |
| 6: 44304547: G > C | rs74950428 | Rare | AARS2; | POLR1C: | NA |
| | | | POLR1C | Intron Variant | |
| | | | | AARS2: Intron | |
| | | | | Variant | |
| 6: 44304587: C > A | rs74964556 | Rare | AARS2; | POLR1C: | NA |
| | | | POLR1C | Intron Variant | |
| | | | | AARS2: | |
| | | | | Intron Variant | |

White, Hispanic (WHS)

| | | | | | |
|---|---|---|---|---|---|
| 12: 47784123: C > T | rs7306788 | Common | HDAC7 | HDAC7: | AMIGO2 |
| | | | | Synonymous | ENDOU |
| | | | | Variant | PCED1B |
| | | | | | TMEM106C |
| | | | | | RAPGEF3 |
| | | | | | SLC48A1 |
| 15: 74195628: A > T | rs971756 | Common | STRA6 | STRA6: | MPI |
| | | | | Missense | SCAMP2 |
| | | | | Variant (L > M) | SEMA7A |
| | | | | | UBL7 |
| 15: 74195505: G > A | rs971757 | Common | STRA6 | STRA6: Intron | MPI |
| | | | | Variant | SCAMP2 |
| | | | | | SEMA7A |
| | | | | | UBL7 |
| 20: 3191844: A > G | rs73075075 | Common | DDRGK1 | DDRGK1: | DNAAF9 |
| | | | | Intron Variant | ITPA |
| | | | | | SLC4A11 |
| 17: 78222788: G > A | rs17882271 | Common | BIRC5 | BIRC5: Intron | TMEM235 |
| | | | | Variant | |
| 3: 8733903: C > T | rs1974763 | Common | CAV3 | CAV3: | NA |
| | | | | Synonymous | |
| | | | | Variant | |
| 20: 63564680: C > T | rs3810487 | Common | HELZ2 | HELZ2: | GMEB2 |
| | | | | Missense | LIME1 |
| | | | | Variant (R > K) | ARFRP1 |
| | | | | | RTEL1 |
| | | | | | ZGPAT |
| | | | | | SLC2A4RG |
| | | | | | STMN3 |
| 15: 83942878: G > A | rs12901723 | Common | ADAMTSL3 | ADAMTSL3: | ALPK3 |
| | | | | Intron Variant | GOLGA6L4 |
| | | | | | HOMER2 |
| | | | | | NMB |
| | | | | | WDR73 |
| 10: 132208002: G > A | rs79220014 | Common | STK32C | STK32C: Non | DPYSL4 |
| | | | | Coding | JAKMIP3 |
| | | | | Transcript | |
| | | | | Variant | |
| 21: 42575315: A > C | rs112406029 | Common | SLC37A1 | SLC37A1: | PDE9A |
| | | | | Intron Variant | RSPH1 |
| | | | | LOC101928212: | SLC37A1 |
| | | | | 2 KB | |
| | | | | Upstream | |
| | | | | Variant | |
| 11: 11956026: T > C | rs2307073 | Common | USP47 | USP47: | DKK3 |
| | | | | Synonymous | |
| | | | | Variant | |
| 6: 44147432: G > A | rs4714759 | Common | TMEM63B | TMEM63B: | MRPL14 |
| | | | | Missense | |
| | | | | Variant (V > M) | |
| | | | | POLR1C: | |
| | | | | Intron Variant | |
| 6: 44151939: A > G | rs3734697 | Common | TMEM63B | TMEM63B: | MRPL14 |
| | | | | Synonymous | |
| | | | | Variant | |
| | | | | POLR1C: | |
| | | | | Intron Variant | |
| 6: 44149927: C > T | rs4714762 | Common | TMEM63B | TMEM63B: | MRPL14 |
| | | | | Synonymous | |
| | | | | Variant | |
| | | | | POLR1C: | |
| | | | | Intron Variant | |
| 6: 41734881: C > A | rs4487571 | Rare | TFEB | TFEB: Intron | NA |
| | | | | Variant | |
| | | | | MIR10398: | |
| | | | | 2 KB Upstream | |
| | | | | Variant | |
| 1: 85124159: A > G | rs17121745 | Common | DNAI3 | DNAI3: | NA |
| | | | | Missense | |
| | | | | Variant (T > A) | |
| 1: 84483058: T > C | rs147475122 | Common | RPF1 | RPF1: Intron | NA |
| | | | | Variant | |
| 19: 7731701: T > G | rs117034642 | Common | CLEC4G | CLEC4G: | CD209 |
| | | | | Synonymous | EVI5L |
| | | | | Variant | CLEC4M |
| | | | | | TRAPPC5 |

White, Non-Hispanic (WNH)

| | | | | | |
|---|---|---|---|---|---|
| 21: 42575315: A > C | rs112406029 | Common | SLC37A1 | SLC37A1: | CBS |
| | | | | Intron Variant | PDE9A |
| | | | | LOC101928212: | RSPH1 |
| | | | | 2 KB | |
| | | | | Upstream | |
| | | | | Variant | |

Following these analyses, additional clinical associations connected with Rare NeanderScores was investigated. Initially, relationships between the different autism-related diagnoses denoted within the SPARK database were examined. Rare Neander-Scores significantly differed by diagnosis in the white non-Hispanic group [F(3, 2000)=2.665, p=0.046], although none of the diagnostic groups significantly differed from each other in Standard post hoc comparison (p=0.156-0.996), making this result difficult to interpret. The other two ethnic groups did not significantly differ in this way (black non-Hispanic: F(3, 68)=2.241, p=0.091; white Hispanic: F(3, 308)=1.915, p=0.127). Next, additional analyses were performed investigating relationships between enriched SNPs that are brain-related QTLs and autism comorbidities. Only one SNP (rs112406029, SLC37A1 host gene) in particular survived multitest correction in the white non-Hispanic group (BH adj. p=0.012) and was significantly associated with epilepsy, occurring in 39% of that sample compared to 26% in non-epileptics and 22% in controls (FIG. 1E, Table 7). In fact, approximately 8% of white non-Hispanic autistic people with seizures were homozygous for this variant, compared to 1.5% in those without epilepsy (X2=17.842, p=2.400×10−5). The rs112406029 enrichment was even more dramatic when family type (multiplex vs. simplex) was added as a covariate [F(1, 1905)=11.055, p=9.014 ×10−4]. Sixty-seven percent of multiplex individuals with epilepsy carried at least one copy of this variant, while 25% of those were homozygous (Table 4, FIG. 1E). Additional clinical associations turned up in the other ethnic groups as well. For instance, in the black non-Hispanic autism group, the rs1805327 missense variant in the RAD1 host gene, which is enriched in this group in general, is over-represented in multiplex cases with language regression (F(1, 66)=8.766, p=0.004; post hoc: pTukey=0.010-0.025). This is in contrast to an inverse relationship between overall Rare NeanderScore and language regression in autistic black non-Hispanics: individuals with higher Rare NeanderScores are significantly less likely to experience language regression (W=348, BH adj. p=0.012). In autistic white Hispanics, almost 80% of males with epilepsy carried the rs2307073 synonymous variant within the epilepsy-associated USP47 host gene, compared to ˜⅓rd of other autistic cases and 15% in the matched controls [F(1, 308)=9.494, p=0.002; post hoc: pTukey=0.008-0.045) [31]. This particular SNP shares LD with additional SNPs within the DKK3 host gene, which is also implicated in the black non-Hispanic group and is involved in regulation of canonical Wnt signaling [32].

TABLE 4
List of Neanderthal-derived brain-related quantitative trait loci (QTL) that share
associations with various autism comorbidities/characteristics by ethnic group.

| | | | | | | Subgroup |
|---|---|---|---|---|---|---|
| Group | Locus | Gene | rs ID | dbSNP | Interaction | enrichment |
| WNH | 21: 42575315: A > C | SLC37A1 | rs112406029 | SLC37A1: Intron | NA | Epilepsy |
| | | | | Variant | | |
| | | | | LOC101928212: | | |
| | | | | 2KB Upstream | | |
| | | | | Variant | | |
| WNH | 21: 42575315: A > C | SLC37A1 | rs112406029 | SLC37A1: Intron | Family type * | Multiplex |
| | | | | Variant | epilepsy | with |
| | | | | LOC101928212: | | epilepsy |
| | | | | 2KB Upstream | | |
| | | | | Variant | | |
| WNH | 15: 52789024: C > A | ONECUT1 | rs2075613 | ONECUT1: | Family type * | Simplex with |
| | | | | Synonymous | epilepsy | epilepsy |
| | | | | Variant | | |
| | | | | LOC105370824: | | |
| | | | | 2KB Upstream | | |
| | | | | Variant | | |
| WNH | 15: 52789024: C > A | ONECUT1 | rs2075613 | ONECUT1: | ID * | ID with |
| | | | | Synonymous | epilepsy | epilepsy |
| | | | | Variant | | |
| | | | | LOC105370824: | | |
| | | | | 2KB Upstream | | |
| | | | | Variant | | |
| WNH | 15: 52789024: C > A | ONECUT1 | rs2075613 | ONECUT1: | Language | Language |
| | | | | Synonymous | delay * | delay with |
| | | | | Variant | epilepsy | epilepsy |
| | | | | LOC105370824: | | |
| | | | | 2KB Upstream | | |
| | | | | Variant | | |
| WNH | 15: 52789024: C > A | ONECUT1 | rs2075613 | ONECUT1: | Language | Language |
| | | | | Synonymous | regression * | regression |
| | | | | Variant | epilepsy | with |
| | | | | LOC105370824: | | epilepsy |
| | | | | 2KB Upstream | | |
| | | | | Variant | | |
| BNH | 3: 128806410: C > T | RAB7A | rs4548 | RAB7A: | Family | Multiplex |
| | | | | Synonymous | Type * | with no |
| | | | | Variant | regression | language |
| | | | | | | regression |
| BNH | 5: 34908772: T > C | RAD1 | rs1805327 | RAD1: Missense | Family | Multiplex |
| | | | | Variant (E > G) | Type * | with |
| | | | | TTC23L: Intron | language | language |
| | | | | Variant | regression | regression |
| BNH | 12: 8857630: A > G | A2ML1 | rs73038782 | A2ML1: Intron | ID * family | Multiplex |
| | | | | Variant | type | with ID |
| BNH | 19: 10328622: A > G | RAVER1 | rs78083518 | RAVER1: Intron | ID * | ID and no |
| | | | | Variant | language | language |
| | | | | | delay | delay |
| BNH | 19: 10328825: C > T | RAVER1 | rs3745263 | RAVER1: Intron | ID * | ID and no |
| | | | | Variant | language | language |
| | | | | | delay | delay |
| WHS | 20: 57330052: G > A | SPO11 | rs28368064 | SPO11: Intron | Family type * | Multiplex |
| | | | | Variant | language | with |
| | | | | LOC105372687: | regression | language |
| | | | | 2KB Upstream | | regression |
| | | | | Variant | | |
| WHS | 20: 57374816: G > T | RAE1 | rs41310034 | RAE1: Intron | Family type * | Multiplex |
| | | | | Variant | language | with |
| | | | | | regression | language |
| | | | | | | regression |
| WHS | 1: 21227230: G > T | ECE1 | rs3026903 | ECE1: Intron | Family type * | Multiplex |
| | | | | Variant | epilepsy | with |
| | | | | | | epilepsy |
| WHS | 20: 57330052: G > A | SPO11 | rs28368064 | SPO11: Intron | ID | ID with |
| | | | | Variant | language | language |
| | | | | LOC105372687: | regression | regression |
| | | | | 2KB Upstream | | |
| | | | | Variant | | |
| WHS | 20: 57374816: G > T | RAE1 | rs41310034 | RAE1: Intron | ID * | ID with |
| | | | | Variant | language | language |
| | | | | | regression | regression |
| WHS | 11: 9021971: A > G | SCUBE2 | rs75002200 | SCUBE2: Intron | Language | Language |
| | | | | Variant | regression * | regression |
| | | | | | epilepsy | with |
| | | | | | | epilepsy |
| WHS | 6: 41734881: C > A | TFEB | rs4487571 | TFEB: Intron | Language | Language |
| | | | | Variant | regression * | regression |
| | | | | MIR10398: 2KB | epilepsy | with |
| | | | | Upstream Variant | | epilepsy |
| WHS | 7: 770427: G > A | DNAAF5 | rs79433478 | DNAAF5: Intron | Sex * | Females |
| | | | | Variant | language | with |
| | | | | | regression | language |
| | | | | | | regression |
| WHS | 7: 869411: G > A | SUN1 | rs61744747 | SUN1: | Sex * | Females |
| | | | | Synonymous | language | with |
| | | | | Variant | regression | language |
| | | | | LOC124901568: | | regression |
| | | | | Intron Variant | | |

This analysis was extended by investigating relationships between clinical phenotypes and rare SNPs by group—particularly those SNPs that are brain-related QTLs. While there were no single phenotype/SNP pairings that survived multitest correction, there were a number of comorbid phenotypes that exhibited significant associations with particular rare SNPs (FIG. 1E, Table 4). A notable example includes the rs20756613 SNP within the ONECUT1 host gene in the white non-Hispanic group and its association with epilepsy and various other clinical features, such as intellectual disability (ID) and language delay. The rs4487571 variant in the TFEB host gene, which is a transcription factor that acts as a master regulator of a variety of metabolic and immune pathways, is enriched in white Hispanics with language regression and epilepsy. Finally, the rs73038782 variant in the A2ML1 host gene, an inhibitor of various proteases and linked with some cases of Noonan syndrome, is enriched in black non-Hispanics from multiplex families without ID.

Neanderthals experienced a prolonged genetic bottleneck, leading to greater retention of nonsynonymous mutations within their dwindling population, suggesting some of these variants may still be represented in the human genome today. Juric et al. found that these weakly deleterious variants, though retained in Neanderthals as a result of small population size, have been under purifying selection once they entered the H. sapiens background with access to a larger population. In support, Wei et al. reported Neanderthal variants are significantly depleted in the modern genome relative to alleles matched for frequency and linkage disequilibrium.

Here is reported an enrichment of a subset of rare and common Neanderthal-derived polymorphisms in autism across three major ethnic groups (black non-Hispanic, white Hispanic, and white non-Hispanic). The low frequency of some of these SNPs, along with their clinical associations, suggests they are weakly deleterious and under continued purifying selection. These trends are not, however, accompanied by a general enrichment in overall Neanderthal content in the SPARK groups, which suggests that not all Neanderthal-derived DNA is equally implicated in susceptibility.

### Neanderthal-Derived Variants in Other Neurodevelopmental Conditions

Previous studies have investigated the potential roles of Neanderthal DNA in susceptibility to other neurodevelopmental conditions. For instance, Gregory et al. reported that lower NeanderScores are associated with schizophrenia and, in particular, with the positive symptoms often present in the condition, suggesting Homo sapiens specific variants, rather than Neanderthal, may be playing a role in susceptibility to the condition. It should be pointed out, however, that these findings do not necessarily disagree with embodiments of the invention. For instance, the present autism samples are not necessarily enriched in all frequency types of Neanderthal variants studied. While white Hispanic autistic people have a dramatic enrichment of rare variants, they have lower total NeanderScores compared to ethnically matched controls. A similar trend is seen in this group and black non-Hispanics when calculating common Neander-Scores, i.e., variants that occur in ≥1% in controls. It is demonstrated that a select subset of Neanderthal-derived variants are playing roles in autism susceptibility and other comorbid features, such as ID, epilepsy, and language regression. A subset of Neanderthal-derived SNPs might not be playing similar roles in schizophrenia susceptibility, despite lower overall NeanderScores.

Along a similar line of research, Srinivasan et al. identified lower rates of Neanderthal Selective Sweeps (Neanderthal variant enriched regions) in portions of the genome that are overrepresented by brain-related genes implicated in schizophrenia. However, it has previously been recognized that brain-related genes, including major effect genes implicated in autism, are tightly conserved and generally mutation intolerant as a result of dosage sensitivity [38]. A similar immutability is seen in schizophrenia-related genes [39]. Therefore, one would expect genomic introgression to be underrepresented in such regions following events such as hybridization. However, even small numbers of key variants in such genes, as a result of their relative intolerance, may nevertheless be keenly felt. In addition, it is vital to understand the contexts under which Homo sapiens-specific variation occurred. Hybridization in non-human species is recognized as a destabilizing event with the potential to promote compensatory adaptation in other regions of the genome and, ultimately, speciation in some lineages [40]. Therefore, there is an additional possibility that Homo sapiens-specific variants that evolved after Neanderthal introgression may nevertheless be influenced by these earlier events.

### Neanderthal DNA, Social Cognition, & Visual Processing

The roles that some Neanderthal-derived variants play in connectivity within the intraparietal sulcus and their concomitant influence on social abilities and visual processing have implications for some of the clinical features of autism. Given the results of the current work, some may ask whether features of autism are reminiscent of behaviors seen in Neanderthal people. Of relevance, recent evidence from numerous individuals from the Altai Mountains of southern Siberia has shown that their genomes held long segments of homozygosity, indicative of small breeding pools. From these and other data, the Applicant makes inferences about Neanderthals' social organization, reinforcing previous notions that they likely lived in small communities. Previous studies have found that social group size of a primate species is strongly predictive of overall neocortical size, suggesting links between group size and cognitive function. While Neanderthals appear to have maintained more regional patterns of social interaction, early European AMH seem to have engaged in broader, better integrated social networks. An interesting topic for future research is whether Neanderthal-derived socio behavioral tendencies may be reflected in some people on the autism spectrum (a topic colloquially known as the “Neanderthal Theory of Autism”).

Conversely, individuals on the autism spectrum often exhibit strengths in visuospatial processing. While an extensive array of tool-making techniques is associated with AMH in the Upper Paleolithic, it should be noted that the Levallois technique, which is most associated with Neanderthal societies in the Middle Paleolithic, is thought to require more skill and training than those necessary to produce the later Upper Paleolithic blades, indicating that Neanderthals were indeed capable of exceptional craftsmanship. Recent research has also revealed additional decorative artistry in Neanderthal societies, with evidence of the use of flight feathers culled from birds of prey for probable personal adornment, as well as early examples of cave art. Relevant to the fields of paleoanthropology and paleoarcheology, there are numerous examples of autistic savants with exceptional visuospatial abilities (e.g., artistic ability) who nevertheless have significant challenges with verbal communication, suggesting that the use of visual symbolism in ancient humans is not necessarily an adequate proxy for presumed language ability as these are modular functions.

Pertinent to the topic of autistic abilities and savantism, although most studies on autism genomics focus on the deleterious nature of variants, there is the possibility some of these autism-associated Neanderthal SNPs have been under weak positive selection. In support, recent studies have identified genetic variants implicated in both autism and high intelligence. Meanwhile, autistic people often perform better on tests of fluid intelligence than neurotypicals. The variable penetrance of these Neanderthal variants for autism, as evidenced by similar patterns of enrichment in unaffected siblings, as well as enrichment of some common variants, suggest a means for their retention. In further support, studies on the cognitive ability of unaffected siblings show sibs tend to have higher performance IQ scores relative to verbal IQ—a pattern very similar to affected siblings and different from neurotypicals. In addition, families of students studying disciplines like math, physics, and engineering are more likely to have autistic family members than students studying the humanities, suggesting an extended cognitive phenotype exists.

Of potential relevance to this topic, paleoarcheologists have identified an “Upper Paleolithic Revolution” beginning around 50 kya, which was a change in the ways AMH made tools, traveled and engaged in trade, used and produced artistic materials, and structured their living habitats, such as dividing the home into food preparation, discard, cooking, and sleeping areas. Interestingly, this time period roughly coincides with hybridization between H. sapiens and Neanderthals, suggesting hybridization may have been a stimulus for cognitive change, one which may continue to influence intellectual ability and susceptibility to neurodevelopmental conditions in modern humans. This has been hypothesized before, although from a sociocultural rather than a genetics perspective.

### Example 4

The following example further analyzes a subset of SNPs from Example 3 with a larger sample size. The subset was the SNPs identified as a set of Neandertal-derived SNPs significantly enriched in Black American autistic probands relative to ethnically-matched non-clinical controls. A replication utilizing a larger cohort was undertaken.

Analyses were performed as in Example 3 except with an expanded cohort using additional Black American autistic samples from the Simons Foundation Powering Autism Research for Knowledge (SPARK) cohort and compared SNP frequencies against the Allele Frequency Aggregator (ALFA) control database, not only for increased statistical power but for valuable cross-validation. Methods for accession of SPARK data were identical to the original publication, while ALFA-based allele frequencies for African Americans, which formed the control group, were accessed from the dbSNP database (Phan et al., 2025, Nucleic Acids Res, 53, D925-D931). Fisher's exact test was used to determine if allele counts differed significantly between SPARK and ALFA for the SNPs of interest, which showed significant enrichment of each of the five SNPs in the autism group (p=8.57×10⁻⁶−3.88×10⁻²⁹) These findings robustly replicate our original results, with several SNPs demonstrating even stronger frequency enrichment in the autism group.

As shown in FIG. 4 and Table 5, these SNPs are uncommon in non-clinical Black American controls (≤2.5%) but notably elevated in autistic individuals (ranging from ˜6% to >17%). These disparities translate into pronounced odds ratios (FIG. 5), supporting their potential clinical utility as ancestry-aware biomarkers.

TABLE 5
SNP Frequencies by Group

| | | Total | | | |
|---|---|---|---|---|---|
| | | SPARK | | ALFA African- | |
| | | Sample | SPARK | American | ALFA |
| Locus | Gene | Size | Frequency | Sample Size | Frequency |
| chr17: 14076741: A > T | COX10 | 349 | 10.32% | 3,452 | 2.32% |
| chr5: 34908772: T > C | RAD1 | 249 | 12.45% | 27,672 | 1.46% |
| chr11: 12008896: C > G | DKK3 | 202 | 15.84% | 2,832 | 2.51% |
| chr10: 132208002: G > A | STK32C | 387 | 17.31% | 2,832 | 2.30% |
| chr6: 44304547: G > C | AARS2; | 218 | 5.96% | 3432 | 1.17% |
| | POLR1C | | | | |

As shown above, five Neandertal-derived SNPs were validated as autism biomarkers in Black American individuals using expanded SPARK autism data and ALFA population controls. Frequencies in autistic probands were 3-10× higher than in ethnically matched controls, with odds ratios confirming strong enrichment. These findings replicate Example 3 and suggest ancestry-sensitive diagnostic utility.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

The above specification provides a description of the manufacture and use of the disclosed compositions and methods. Since many embodiments can be made without departing from the spirit and scope of the disclosure, the disclosure resides in the claims.

1. A method of diagnosing autism, the method comprising:
determining the levels of one or more biomarkers in a biological sample from a subject, wherein the one or more biomarkers comprise one or more single nucleotide polymorphisms (SNPs) found in Cox10, RADI, DKK3, STK32C, AARS2, POLRIC, or a combination thereof,
wherein elevated levels of the one or more biomarkers is an indicator of autism in the subject.

2. The method of claim 1, wherein the two or more biomarkers comprises an SNP from found in Cox10, RADI, DKK3, STK32C, AARS2 and POLRIC.

3. The method of claim 1, wherein the three or more biomarkers comprises an SNP found in Cox10, RADI, DKK3, STK32C, AARS2 and POLRIC.

4. The method of claim 1, wherein the one or more SNPs are found in each of Cox10, RADI, DKK3, STK32C, AARS2 and POLRIC.

5. The method of claim 1, wherein the subject is European American.

6. The method of claim 1, wherein the subject is African American.

7. A method of identifying elevated levels of a biomarker, the method comprising:
determining the levels of one or more biomarkers in a biological sample from a subject, wherein the one or more indicators comprises wherein the one or more biomarkers comprise one or more single nucleotide polymorphisms (SNPs) found in Cox10, RADI, DKK3, STK32C, AARS2, POLRIC, or a combination thereof,
wherein elevated levels of the one or more biomarkers is an indicator of autism in the subject.

8. The method of claim 7, wherein the one or more biomarkers comprises an SNP from each of Cox10, RADI, DKK3, STK32C, AARS2 and POLRIC.

9. The method of claim 8, wherein the subject is European American.

10. The method of claim 9, wherein the subject is African American.

11. A gene panel for diagnosing autism spectrum disorder (ASD) in a subject, the panel comprising a plurality of nucleic acid markers selected from the group consisting of COX10, RAD1, DKK3, STK32C, AARS2, and POLRIC, wherein the panel comprises at least two of said markers, and wherein detection of genetic variation in said markers is indicative of a predisposition to or diagnosis of ASD.

12. (canceled)
